SEQUENCE LISTING (10 sequences)

SEQ ID NO: 1 - DNA, 25 bp, Agrobacterium tumefaciens
tggcaggatatataccgttgtaatt

SEQ ID NO: 2 - DNA, 25 bp, Agrobacterium tumefaciens
cggcaggatatattcaattgtaaat

SEQ ID NO: 3 - DNA, 25 bp, Agrobacterium tumefaciens
cggcaggatatattcaattgtaaac

SEQ ID NO: 4 - DNA, 25 bp, Agrobacterium tumefaciens
tgacaggatatattggcgggtaaac

SEQ ID NO: 5 - DNA, 25 bp, Agrobacterium tumefaciens
tggcaggatatattgtggtgtaaac

SEQ ID NO: 6 - DNA, 25 bp, Agrobacterium tumefaciens
tggcaggatatatcgaggtgtaaaa

SEQ ID NO: 7 - DNA, 25 bp, Agrobacterium tumefaciens
tggcaggatatatgcggttgtaatt

SEQ ID NO: 8 - DNA, 25 bp, Agrobacterium rhizogenes
tgacaggatatatccccttgtctag

SEQ ID NO: 9 - DNA, 10 bp, Artificial Sequence
  consensus
caggatatat

SEQ ID NO: 10 - protein, 9 aa, Artificial Sequence
  consensus
LAGLIDADG

(19) United States
(12) Patent Application Publication
(10) Pub. No.: US 2016/0222395 A1
(43) Pub. Date: Aug. 4, 2016

(54) AGROBACTERIUM-MEDIATED GENOME MODIFICATION WITHOUT T-DNA INTEGRATION

(71) Applicant: CELLECTIS, Paris (FR)

(72) Inventors: Thomas Stoddard, St. Louis Park, MN (US); Nicholas J. Baltes, New Brighton, MN (US); Song Luo, Chicago, IL (US)

(21) Appl. No.: 15/013,323

(22) Filed: Feb. 2, 2016

Related U.S. Application Data
(60) Provisional application No. 62/110,735, filed on Feb. 2, 2015.

(51) Int. Cl. C12N 15/82

(52) U.S. Cl. CPC C12N 15/8205

(57) ABSTRACT

Methods for genome engineering, including methods utilizing transient expression of a nuclease utilizing modified transfer-DNA (T-DNA) plasmids, are provided herein.

### CROSS-REFERENCE TO RELATED APPLICATIONS

This application claims benefit of priority from U.S. Provisional Application Ser. No. 62/110,735, filed on Feb. 2, 2015, which is incorporated here by reference in its entirety.

### TECHNICAL FIELD

This invention relates to methods for genome engineering, including methods for genome engineering through transient expression of a nuclease utilizing modified transfer-DNA (T-DNA) plasmids.

### BACKGROUND

Agrobacterium is a genus of Gram-negative bacteria that uses horizontal gene transfer to cause tumorigenesis in plants via the introduction of transfer DNA (T-DNA) into the plant genome via large tumor-inducing (Ti) or rhizogenic (Ri) plasmids. To be virulent, Agrobacterium must contain a Ti or Ri plasmid that has the T-DNA and all the genes necessary to transfer the T-DNA to the plant cell and integrate it into the chromosomal DNA. Although there are variations of both Ti and Ri plasmids, several features are common among naturally occurring strains: virulence genes, an origin of replication, opine catabolism genes, a right border (RB) sequence, a left border (LB) sequence, and a transfer DNA (T-DNA) region. The virulence genes and border sequences allow the Agrobacterium to transfer the T-DNA into a plant cell via a type IV secretion system (TIVSS). Once the T-DNA is transformed into the plant cell, it is capable of integrating into the host genome with the help of the Agrobacterium virulence proteins. The integrated T-DNA may contain oncogenic and opine synthesis genes that allow for increased production of opines, which act as the Agrobacterium's source of carbon and nitrogen. The Ti and Ri plasmids are significantly different at the nucleotide level, yet the plasmids can be exchanged between A. tumefaciens and A. rhizogenes, thus granting the bacterium a new pathogenic profile indicative of the Ti or Ri plasmid it contains. Agrobacterium T-DNA can be modified and used in binary vector systems, with virulence genes and T-DNA on separate plasmids. This strategy has been used to introduce new genes into plant genomes (see, for example, Lee and Gelvin, Plant Physiol 146:325-332, 2008). The virulence genes on the Ti or Ri plasmid and many Agrobacterium chromosomal genes are deemed essential to the mechanism of integration. However, the mechanism of integration has not been completely elucidated.

### SUMMARY

The present document is based in part on the discovery that Agrobacterium-mediated transformation can be used for transient expression of sequence-specific nucleases in plant cells, to yield genetically modified plants that are non-transgenic. For example, Agrobacterium can be used to introduce T-DNA encoding a desired nuclease gene into plant cells, allowing for expression of the nuclease without T-DNA integration. The transient expression of such nucleases can result in site-directed genome modification, enabling precise engineering of the chosen plant species. This can eliminate the need for subsequent backcrossing to remove foreign DNA integrated by traditional Agrobacterium transformation, reduce regulatory concerns, and increase the speed to market.

This document features, inter alia, a method for transiently expressing a polypeptide in a plant cell. The method can include introducing into a plant cell a modified Ti, Ri, or T-DNA plasmid containing a T-DNA region that includes (a) a T-DNA border sequence, and (b) a polypeptide-encoding sequence containing a 5′ promoter region, a structural coding sequence encoding a polypeptide, and a 3′ non-translated region encoding a polyadenylation signal, where the 5′ promoter region and the 3′ non-translated region are operably linked to the structural coding sequence, such that the polypeptide encoding sequence is transiently expressed in the plant cell and does not integrate into the genome of the plant cell. For example, the method can include introducing into the cell an integration-inhibited T-DNA (iiT-DNA) plasmid corresponding to a Ti, Ri, and T-DNA plasmid that has been modified by removal or inactivation of at least one T-DNA border, such that the integration of the resulting iiT-DNA is reduced. In some embodiments, the LB of the modified Ti plasmid, Ri plasmid, or T-DNA plasmid is removed or inactivated, such that T-DNA integration into the plant genome is impaired. The modified Ti, Ri, or T-DNA plasmid can have at least one T-DNA border sequence that is not functional (e.g., can have only one functional T-DNA border sequence, or can have no functional T-DNA border sequence). In some embodiments, the RB of the iiT-DNA plasmid (containing no LB or an inactivated LB) is rendered removable or inactive once the iiT-DNA has entered a plant cell, such that T-DNA integration is further impaired. The plasmid in such embodiments is designated herein as a removable right border iiT-DNA (RRBiiT-DNA) plasmid. As described herein, a RRBiiT-DNA can be obtained by removal of the RB sequence by a rare cutting endonuclease. In general, the rare cutting endonuclease can be encoded by one of the structural coding sequences included in the T-DNA sequence contained within the modified Ti, Ri, or T-DNA plasmid.

This document also features a method for using a modified Ti, Ri, or T-DNA plasmid as described herein to perform gene editing in a plant cell, without T-DNA integration, where a rare cutting endonuclease is transiently expressed from the plasmid. The rare-cutting endonuclease can be directed against a specific locus in the plant genome, and its action can result in mutation, modification, or repair of the genetic sequences at the specific locus.

In some embodiments, methods can include introducing to the cell (or contacting the cell with) an organism capable of horizontal gene transfer, where the organism contains the modified Ti, Ri, or T-DNA plasmid. The organism capable of horizontal gene transfer in the methods provided herein can be a bacterium (e.g., an Agrobacterium). The T-DNA border sequence can be from Agrobacterium. The iiT-DNA border sequence referred to above can be a T-DNA RB sequence (e.g., an RB sequence from an octopine Ti plasmid, a nopaline Ti plasmid, or an agropine Ti plasmid). The iiT-DNA border sequence can be 5′ of the polypeptide-encoding sequence in the Ti or Ri plasmid. The 5′ promoter region can exist naturally in a plant cell, or can be capable of naturally entering a plant cell. The 5′ promoter region can include a constitutive promoter, or the 5′ promoter region can include an inducible promoter and the method can further include inducing the promoter. The polypeptide-encoding sequence can encode a rare-cutting endonuclease or rare-cutting endonuclease subunit. The rare-cutting endonuclease can be a transcription activator-like (TAL) effector endonuclease (also referred to as a TALE nuclease or TALEN®), a zinc-finger nuclease, a meganuclease, or a programmable RNA-guided endonuclease. Transient expression of the rare-cutting endonuclease can result in site-directed mutagenesis. The modified Ti, Ri, or T-DNA plasmid can contain a reporter gene that is transiently expressed with the structural coding sequence. Expression of the reporter gene can result in a visual signal or antibiotic resistance. The T-DNA region can further include a donor sequence. Transient delivery of the donor sequence to the cell can result in gene targeting.

The T-DNA region can further contain a second polypeptide-encoding sequence having a 5′ promoter region, a structural coding sequence encoding a second polypeptide, and a 3′ non-translated region encoding a polyadenylation signal, where the 5′ promoter region and the 3′ non-translated region are operably linked to the structural coding sequence, such that the second polypeptide-encoding sequence is transiently expressed in the plant cell and does not integrate into the genome of the plant cell. The 5′ promoter region of the second polypeptide-encoding sequence can exist naturally in a plant cell or can be capable of naturally entering a plant cell. The 5′ promoter region can include a constitutive promoter, or the 5′ promoter region can include an inducible promoter and the method can further include inducing the promoter. The polypeptide-encoding sequence can encodes a rare-cutting endonuclease (e.g., a TAL effector endonuclease, a zinc-finger nuclease, a meganuclease, or a programmable RNA-guided endonuclease) or rare-cutting endonuclease subunit, and the second polypeptide-encoding sequence can encode a rare-cutting endonuclease or rare-cutting endonuclease subunit. Transient expression of the rare-cutting endonuclease or rare-cutting endonuclease subunits can result in site-directed mutagenesis.

The T-DNA can further contain a duplicated and inverted sequence. For example, the T-DNA can include a duplicated and inverted sequence that is within about 1000 nucleotides of the T-DNA border sequence, such as within about 500 to 1000 nucleotides of the T-DNA border sequence, within about 250 to 500 nucleotides of the T-DNA border sequence, or within about 1 to 500 nucleotides of the T-DNA border sequence. In some embodiments, the duplicated and inverted sequence can be at the border sequence, such that the duplicated sequence contains a border (e.g., a border rendered nonfunctional due to mutation) and additional T-DNA. In some embodiments, the duplicated and inverted sequence can be adjacent to the border sequence, but not include the border sequence. In both cases, the duplicated and inverted sequence can facilitate the forming of a stem-loop structure at one end of the linear T-DNA molecule.

In another aspect, this document features a method for generating a plant. The method can include (a) providing a plant cell obtained according to a method that includes introducing a susceptible plant cell to an organism capable of horizontal gene transfer, where the organism contains a modified Ti, Ri, or T-DNA plasmid containing a T-DNA region that includes (i) a T-DNA border sequence, and (ii) a polypeptide-encoding sequence containing a 5′ promoter region, a structural coding sequence encoding a polypeptide, and a 3′ non-translated region encoding a polyadenylation signal, where the 5′ promoter region and the 3′ non-translated region are operably linked to the structural coding sequence, such that the polypeptide-encoding sequence is transiently expressed in the plant cell and does not integrate into the genome of the plant cell, and where the polypeptide-encoding sequence encodes a rare-cutting endonuclease or a rare-cutting endonuclease subunit, and (b) regenerating the plant cell into a plant. The regenerated plant can contain one or more mutations generated by transient expression of the rare-cutting endonuclease.

In another aspect, this document features a method for generating a plant. The method can include (a) providing a plant cell obtained according to a method that includes introducing a susceptible plant cell to an organism capable of horizontal gene transfer, where the organism contains a modified Ti, Ri, or T-DNA plasmid that contains a T-DNA region that includes (i) a T-DNA border sequence, (ii) a polypeptide-encoding sequence containing a 5′ promoter region, a structural coding sequence encoding a polypeptide, and a 3′ non-translated region encoding a polyadenylation signal, where the 5′ promoter region and the 3′ non-translated region are operably linked to the structural coding sequence, and (iii) a second polypeptide-encoding sequence containing a 5′ promoter region, a structural coding sequence encoding a second polypeptide, and a 3′ non-translated region encoding a polyadenylation signal, where the 5′ promoter region and the 3′ non-translated region are operably linked to the structural coding sequence, such that the polypeptide-encoding sequences are transiently expressed in the plant cell and do not integrate into the genome of the plant cell, and where the polypeptide-encoding sequence encodes a rare-cutting endonuclease or rare-cutting endonuclease subunit, and the second polypeptide-encoding sequence can encode a rare-cutting endonuclease or rare-cutting endonuclease subunit, and (b) regenerating the plant cell into a plant. The regenerated plant can contain one or more mutations generated by transient expression of the rare-cutting endonucleases or rare-cutting endonuclease subunits.

In another aspect, this document features a method for transiently expressing a polypeptide in a plant cell, where the method includes introducing a plant cell to an organism capable of horizontal gene transfer, where the organism contains a modified Ti, Ri, or T-DNA plasmid having a T-DNA region that includes (a) a T-DNA border sequence, (b) a target site for a rare-cutting endonuclease, and (c) a polypeptide-encoding sequence including a 5′ promoter region, a structural coding sequence encoding a polypeptide, and a 3′ non-translated region encoding a polyadenylation signal, where the 5′ promoter region and the 3′ non-translated region are operably linked to the structural coding sequence, such that the polypeptide-encoding sequence is transiently expressed in the plant cell and does not integrate into the genome of the plant cell. The organism capable of horizontal gene transfer can be a bacterium (e.g., an Agrobacterium). The T-DNA border sequence can be from Agrobacterium. The T-DNA border sequence can be a T-DNA right border sequence. The T-DNA border sequence can be from an octopine Ti plasmid, a nopaline Ti plasmid, or an agropine Ti plasmid. The T-DNA border sequence can be 5′ of the polypeptide-encoding sequence in the Ti or Ri plasmid. The 5′ promoter region can exist naturally in a plant cell, or can be capable of naturally entering a plant cell. The 5′ promoter region can include a constitutive promoter, or the 5′ promoter region can include an inducible promoter, and the method can further include inducing the promoter. The polypeptide-encoding sequence can encode a rare-cutting endonuclease or rare-cutting endonuclease subunit (e.g., TAL effector endonuclease, a zinc-finger nuclease, a meganuclease, or a programmable RNA-guided endonuclease). Transient expression of the rare-cutting endonuclease can result in site-directed mutagenesis.

In another aspect, this document features a method for transiently expressing a polypeptide in a plant cell, where the method includes contacting a plant cell with an organism capable of horizontal gene transfer, where the organism contains a modified Ti, Ri, or T-DNA plasmid having a T-DNA region that includes (a) a T-DNA border sequence, and (b) a polypeptide-encoding sequence that includes a 5′ promoter region, a structural sequence encoding the polypeptide, and a 3′ non-translated region containing a polyadenylation signal, where the 5′ promoter region and the 3′ non-translated region are operably linked to the structural coding sequence. The T-DNA also can include (c) a target site for a rare-cutting endonuclease. The polypeptide-encoding sequence can encode a rare-cutting endonuclease or rare-cutting endonuclease subunit that specifically recognizes and cleaves DNA at its target site (e.g., at the target site included in the T-DNA). For example, expression of the rare-cutting endonuclease can result in a double-stranded break of the rare-cutting endonuclease target site, removing the T-DNA border. Without being bound by a particular theory, removal of the T-DNA border also may entail the removal of proteins that are covalently attached to the target site, which may drive the T-DNA toward random insertion into plant chromosomal DNA.

The modified Ti, Ri, or T-DNA plasmid also may include a reporter gene that is transiently expressed with the structural coding sequence. Expression of the reporter gene can result in a visual signal or antibiotic resistance. In some embodiments, the same rare-cutting endonuclease encoded by the polypeptide-encoding sequence included in the T-DNA can cleave both the rare-cutting endonuclease target sequence located in the T-DNA plasmid and the genomic target DNA in the plant genome.

The T-DNA region can further include a second polypeptide-encoding sequence having a 5′ promoter region, a structural coding sequence encoding a second polypeptide, and a 3′ non-translated region encoding a polyadenylation signal, where the 5′ promoter region and the 3′ non-translated region are operably linked to the structural coding sequence, such that the second polypeptide-encoding sequence is transiently expressed in the plant cell and does not integrate into the genome of the plant cell. The 5′ promoter region of the second polypeptide-encoding sequence can exist naturally in a plant cell, or can be capable of naturally entering a plant cell. The 5′ promoter region can include a constitutive promoter, or the 5′ promoter region can include an inducible promoter and the method can further include inducing the promoter. The polypeptide-encoding sequence can encode a rare-cutting endonuclease or rare-cutting endonuclease subunit, and the second polypeptide-encoding sequence can encode a rare-cutting endonuclease or rare-cutting endonuclease subunit. The rare-cutting endonuclease can be a TAL effector endonuclease, a zinc-finger nuclease, a meganuclease, or a programmable RNA-guided endonuclease. Transient expression of the rare-cutting endonuclease or rare-cutting endonuclease subunits can result in site-directed mutagenesis.

The method can further include introducing the plant cell to a second organism capable of horizontal gene transfer, where the second organism contains a modified Ti, Ri, or T-DNA plasmid having a T-DNA region that includes a T-DNA border sequence, a second polypeptide-encoding sequence containing a 5′ promoter region, a structural coding sequence encoding a polypeptide, and a 3′ non-translated region encoding a polyadenylation signal, where the 5′ promoter region and the 3′ non-translated region are operably linked to the structural coding sequence, such that the second polypeptide encoding sequence is transiently expressed in the plant cell and does not integrate into the genome of the plant cell. The second organism can be introduced to the plant cell within five days of the first organism. The 5′ promoter region of the polypeptide-encoding sequence and the 5′ promoter region of the second polypeptide-encoding sequence can exist naturally in a plant cell, or can be capable of naturally entering a plant cell. The 5′ promoter region can include a constitutive promoter, or the 5′ promoter region can include an inducible promoter and the method can further include inducing the promoter. The polypeptide-encoding sequence can encode a rare-cutting endonuclease or rare-cutting endonuclease subunit, and the second polypeptide-encoding sequence can encode a rare-cutting endonuclease or rare-cutting endonuclease subunit. The rare-cutting endonuclease can be a TAL effector endonuclease, a zinc-finger nuclease, a meganuclease, or a programmable RNA-guided endonuclease. Transient expression of the rare-cutting endonucleases or rare-cutting endonuclease subunits can result in site directed mutagenesis. Expression of the rare-cutting endonuclease or rare-cutting endonuclease subunits can result in a double-stranded break of the rare-cutting endonuclease target site, removing the first T-DNA border and covalently attached proteins. The T-DNA region can further include a donor sequence. Transient delivery of the donor sequence to the cell can result in gene targeting.

The method can further include introducing to the plant cell a second organism capable of horizontal gene transfer, where the second organism contains a modified Ti, Ri, or T-DNA plasmid having a T-DNA region that includes a T-DNA border sequence, a second polypeptide-encoding sequence containing a 5′ promoter region, a structural coding sequence encoding a polypeptide, and a 3′ non-translated region encoding a polyadenylation signal, where the 5′ promoter region and the 3′ non-translated region are operably linked to the structural coding sequence; and a third polypeptide-encoding sequence containing a 5′ promoter region, a structural coding sequence encoding a polypeptide, and a 3′ non-translated region encoding a polyadenylation signal, where the 5′ promoter region and the 3′ non-translated region are operably linked to the structural coding sequence, such that the second and third polypeptide-encoding sequences are transiently expressed in the plant cell and are not integrated into the genome of the plant cell. The second organism can be introduced to the plant cell within five days of the first organism. The second polypeptide-encoding sequence can encode a rare-cutting endonuclease or rare-cutting endonuclease subunit, and the third polypeptide-encoding sequence encodes a rare-cutting endonuclease or rare-cutting endonuclease subunit. The rare-cutting endonuclease can be a TAL effector endonuclease, a zinc-finger nuclease, a meganuclease, or a programmable RNA-guided endonuclease. Transient expression of the rare-cutting endonucleases or rare-cutting endonuclease subunits can result in site-directed mutagenesis. The T-DNA region can further include a donor sequence. Transient delivery of the donor sequence can result in gene targeting. Expression of the rare-cutting endonuclease or rare-cutting endonuclease subunits can result in a double-stranded break of the rare-cutting endonuclease target site, removing the first T-DNA border and covalently attached proteins.

In another aspect, this document features a method for generating a plant, where the method includes (a) providing a plant cell obtained according to a method that includes introducing a plant cell to an organism capable of horizontal gene transfer, where the organism contains a modified Ti, Ri, or T-DNA plasmid having a T-DNA region that includes (i) a T-DNA border sequence, (ii) a target site for a rare-cutting endonuclease, and (iii) a polypeptide-encoding sequence containing a 5′ promoter region, a structural coding sequence encoding a polypeptide, and a 3′ non-translated region encoding a polyadenylation signal, where the 5′ promoter region and the 3′ non-translated region are operably linked to the structural coding sequence, such that the polypeptide-encoding sequence is transiently expressed in the plant cell and does not integrate into the genome of the plant cell, and where the polypeptide-encoding sequence encodes a rare-cutting endonuclease or a rare-cutting endonuclease subunit, and (b) regenerating the plant cell into a plant. The regenerated plant can contain one or more mutations generated by transient expression of the rare-cutting endonuclease.

In still another aspect, this document features a method for generating a plant, where the method includes (a) providing a plant cell obtained according to a method that includes introducing a plant cell to an organism capable of horizontal gene transfer, where the organism contains a modified Ti, Ri, or T-DNA plasmid having a T-DNA region that includes (i) a T-DNA border sequence, (ii) a target site for a rare-cutting endonuclease, (iii) a polypeptide-encoding sequence containing a 5′ promoter region, a structural coding sequence encoding a polypeptide, and a 3′ non-translated region encoding a polyadenylation signal, where the 5′ promoter region and the 3′ non-translated region are operably linked to the structural coding sequence, and (iv) a second polypeptide-encoding sequence containing a 5′ promoter region, a structural coding sequence encoding a second polypeptide, and a 3′ non-translated region encoding a polyadenylation signal, where the 5′ promoter region and the 3′ non-translated region are operably linked to the structural coding sequence, such that the polypeptide-encoding sequence is transiently expressed in the plant cell and does not integrate into the genome of the plant cell, and where the polypeptide-encoding sequence encodes a rare-cutting endonuclease or rare-cutting endonuclease subunit, and the second polypeptide-encoding sequence encodes a rare-cutting endonuclease or rare-cutting endonuclease subunit, and (b) regenerating the plant cell into a plant. The regenerated plant can contain one or more mutations generated by transient expression of the rare-cutting endonucleases or rare-cutting endonuclease subunits.

This document also features a modified Ti, Ri, or T-DNA plasmid containing a T-DNA region that includes (a) one T-DNA border sequence, and (b) a polynucleotide sequence encoding a rare-cutting endonuclease or one or more rare-cutting endonuclease subunits, operably linked to a promoter induced in a plant cell. The T-DNA can contain a duplicated and inverted sequence (e.g., a duplicated and inverted sequence adjacent to the border sequence). The rare-cutting endonuclease or rare-cutting endonuclease subunits can be from a TAL effector endonuclease, a zinc-finger nuclease, a meganuclease, or a programmable RNA-guided endonuclease. The modified Ti, Ri, or T-DNA plasmid can further contain a target site for the rare-cutting endonuclease, where the target site is downstream of the T-DNA border sequence.

In addition, this document features an article of manufacture that includes a modified Ti, Ri, or T-DNA plasmid as provided herein.

This document also features a composition that includes a modified Ti, Ri, or T-DNA plasmid as provided herein.

Further, this document features an isolated host cell transformed with a modified Ti, Ri, or T-DNA plasmid as provided herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram of the pCAMBIA-1300 vector.

FIG. 2 is a diagram of a synthesized cassette.

FIG. 3 is a diagram of an iiT-DNA plasmid containing two ALS2 T1 TALE nuclease subunits.

FIG. 4 is an illustration of an iiT-DNA plasmid with duplicated and inverted sequences at the T-DNA border. Also shown are the single-stranded and double-stranded linear T-DNA molecules with predicted hairpin structures.

FIG. 5 is a gel image showing the frequency of mutagenesis of the ALS2 T1 TALE nuclease subunits after being delivered to Nicotiana benthamiana leaves by agroinfiltration. The TALE nuclease subunits were delivered to plant cells using iiT-DNA or conventional T-DNA.

FIG. 6 is a diagram of an RRBiiT-DNA plasmid containing two ALS2 T1 TALE nuclease subunits, and the ALS2 TALEN target site near the RB sequence.

### DETAILED DESCRIPTION

Genetically modified crops offer a route to develop novel plant varieties that are able to thrive under environmental and agricultural constraints, optimizing the energy returned on investment. Transgenic plants typically are generated via the insertion of foreign genetic material, but such methods can require long and arduous regulatory steps before public use is approved. The materials and methods provided herein can be used to generate genetically modified plants that are non-transgenic, thus avoiding at least some of the regulatory steps required for approval for public use. In general, the methods described herein involve transient expression of desired nucleic acids (e.g., nucleic acids encoding nucleases or subunits thereof) via Agrobacterium, which provides a delivery system that can allow for genome engineering without integration of foreign nucleic acids.

To be transferred into a plant cell, the T-DNA generally is first processed from the circular Ti or Ri plasmid. A VirD1/D2 complex binds to and nicks the Ti or Ri DNA at the LB and RB sequences of the T-DNA. These border sequences usually are about 25 bp in length and are repeated in direct orientation, flanking the T-DNA region of the Ti or Ri plasmid (see, e.g., Wang et al., Cell 38:455-462, 1984). The right and left borders delineating the T-DNA region share a low degree of homology among the biovars found in nature, with the most divergent borders sharing about 50% sequence identity, although some share about 80% or more (e.g., about 90% or about 95%) sequence identity. In general, the T-DNA borders include 10 to 13 nucleotides, some containing a conserved CAGGATATAT (SEQ ID NO:13) consensus sequence as shown in Table 1 (see, also, Slightom et al., EMBO J 4(12):3069-3077, 1985).

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\B12seq c:\seq1.txt -j c:\seg2.txt -p blastn -o c:\output.txt -q -1 -r 2. To compare two amino acid sequences, the options of Bl2seq are set as follows: -i is set to a file containing the first amino acid sequence to be compared (e.g., C: \seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq c:\seq1.txt -j c:\seg2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:1), or by an articulated length (e.g., 100 consecutive nucleotides or amino acid residues from a sequence set forth in an identified sequence), followed by multiplying the resulting value by 100. For example, a nucleic acid sequence that has 23 matches when aligned with the sequence set forth in SEQ ID NO:1 is 92 percent identical to the sequence set forth in SEQ ID NO:1 (i.e., 23±25×100=92). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

As described herein, a Ti or Ri plasmid can be a single plasmid that contains the T-region and the virulence genes necessary to export the T-DNA from the bacterium to the plant cell. In some embodiments, a Ti or Ri plasmid can be a T-DNA binary-vector system that includes two plasmids: (i) a helper plasmid that contains the virulence genes necessary for T-DNA processing and transfer to the plant cell, and (ii) the binary vector that contains the T-region. The T-DNA binary vector is referred to herein as the T-DNA plasmid. In some embodiments, a Ti, Ri, or T-DNA plasmid can be the integration of one or both of the necessary virulence genes and T-region into the Agrobacterium chromosomal DNA.

As described herein, Ti, Ri, or T-DNA plasmids can be converted into transient expression plasmids by removal or mutation of a T-DNA border (e.g., the left T-DNA border) such that only one T-DNA border is functional. Such removal or mutation of a T-DNA border eliminates one of the two VirD1/VirD2 endonuclease target sites and thus inhibits normal T-strand formation, which can result in delivery of the entire plasmid backbone to the plant cell.

As used herein, the term “integration-inhibited T-DNA (iiT-DNA) plasmid” refers to a Ti, Ri, or T-DNA plasmid that has been modified by removal or mutation of a T-DNA border, such that T-DNA integration is inhibited. The term iiT-DNA refers to the T-DNA sequence within a Ti, Ri, or T-DNA plasmid that has been modified by removal or mutation of a T-DNA border. In some embodiments, for example, the LB of the iiT-DNA can be removed.

As used herein, the term “removable right border iiT-DNA (RRBiiT-DNA) plasmid” refers to a Ti, Ri, or T-DNA plasmid that has been modified by removal or mutation of a first T-DNA border (e.g., the LB), and has been further modified by the addition of a rare-cutting endonuclease target sequence for the purpose of removing the second border (e.g., the RB). In some embodiments of the materials and methods provided herein, the T-DNA region to be delivered to a plant cell can contain a single functional T-DNA border sequence, as well as one or more (e.g., one, two, three, four, five, or more than five) sequences encoding one or more polypeptides of interest. Thus, the T-DNA region may contain one, and no more than one, T-DNA border sequence that can be nicked by a VirD1/D2 complex. It is to be noted that a T-DNA region may contain one or more additional T-DNA border sequences that are non-functional, such that they are not able to be nicked by a VirD1/D2 complex. Such non-functional T-DNA border sequences can be generated by, for example, mutation of a naturally occurring T-DNA border sequence (e.g., by substituting or disrupting the sequence within the conserved region indicated in Table 1). It is further to be noted that a non-functional T-DNA border sequence may still be bound by a VirD1/D2 complex. Without being bound by a particular mechanism, it is possible that a T-DNA region containing multiple T-DNA border sequences that can be bound by VirD1/D2 complexes may be more effectively transferred into the nucleus.

The functional T-DNA border sequence can be located 5′ of the polypeptide-encoding sequence(s), or 3′ of the polypeptide-encoding sequence(s). In some embodiments, the T-DNA border and the polypeptide-encoding sequence can be immediately adjacent to one another. Alternatively, the T-DNA border and the polypeptide-encoding sequence can be separated by a spacer sequence of about three to about 2000 nucleotides (e.g., about 10 to about 1000 nucleotides, about 10 to about 200 nucleotides, or about 20 to about 100 nucleotides). In some embodiments, when multiple T-DNA border sequences (e.g., multiple RB sequences) are included, they can be clustered, such that they are all 5′ or all 3′ of the polypeptide-encoding sequence(s). It is to be noted, however, that in some embodiments, a T-DNA region can include a functional T-DNA border sequence on one side (e.g., 5′) of the polypeptide-encoding sequence(s), and a non-functional T-DNA border sequence on the other side (e.g., 3′) of the polypeptide-encoding sequence(s).

In some embodiments, a T-DNA border sequence contained within a modified Ti, Ri, or T-DNA plasmid as provided herein can be a RB sequence. For example, the T-DNA border sequence can be a RB sequence from A. tumefaciens or from A. rhizogenes. In some embodiments, the T-DNA border sequence can be a RB sequence from an A. tumefaciens octopine Ti plasmid, a RB sequence from an A. tumefaciens nopaline Ti plasmid, or a RB sequence from an A. rhizogenes agropine Ti plasmid. A list of representative T-DNA border sequences is provided in Table 1. In some embodiments, a functional T-DNA border sequence can be a variant of a sequence as set forth in Table 1, such that the T-DNA border sequence has five or less (e.g., five, four, three, two, or one) additions, subtractions, or substitutions with regard to the corresponding sequence within Table 1. It is again noted that the nucleotides at certain positions are conserved within the T-DNA sequences set forth in Table 1, and thus, the nucleotides at those positions typically are retained in the functional T-DNA border sequences of the constructs provided herein. In some embodiments, however, a functional T-DNA border sequence can have a mutation at one or two of the conserved positions, such that at least 80% (e.g., at least 80% or at least 90%) of the nucleotides at the conserved positions are retained. Further, a non-functional T-DNA border sequence can include mutations within the conserved region that result in loss of the ability to be nicked by the VirD1/D2 complex. Such border sequences may include mutations at, for example, three or more (e.g., three, four, five, six, seven, or more than seven) of the conserved positions.

TABLE 1
T-DNA Border Sequences

| SEQ | | |
|---|---|---|
| ID NO: | Sequence | Description |
| 1 | TGGCAGGATATAT | Octopine pTiAch5 |
| | ACCGTTGTAATT | right |
| 1 | TGGCAGGATATAT | Octopine pTil5955 |
| | ACCGTTGTAATT | left |
| 2 | CGGCAGGATATAT | Octopine pTiA6 |
| | TCAATTGTAAAT | left |
| 2 | CGGCAGGATATAT | Octopine pTiAch5 |
| | TCAATTGTAAAT | left |
| 3 | CGGCAGGATATAT | Octopine pTil5955 |
| | TCAATTGTAAAC | left |
| 4 | TGACAGGATATAT | Nopaline pTiT37 |
| | TGGCGGGTAAAC | right |
| 4 | TGACAGGATATAT | Nopaline pTiT37 |
| | TGGCGGGTAAAC | right |
| 5 | TGGCAGGATATAT | Nopaline pTiT37 |
| | TGTGGTGTAAAC | left |
| 5 | TGGCAGGATATAT | Nopaline pTiT37 |
| | TGTGGTGTAAAC | left |
| 6 | TGGCAGGATATAT | Octopine pTil5955 |
| | CGAGGTGTAAAA | right |
| 7 | TGGCAGGATATAT | Octopine pTil5955 |
| | GCGGTTGTAATT | right |
| 8 | TGACAGGATATAT | K599 Ri plasmid |
| | CCCCTTGTCTAG | right |
| 9 | -G-CAGGATATAT | Consensus* |
| | ------GT---- | |

*indicates nucleotides that are conserved within SEQ ID NOS: 1-12.

The polypeptide-encoding sequence can include a structural coding sequence that encodes the polypeptide of interest, as well as a 5′ promoter region and a 3′ non-translated region encoding a polyadenylation signal, each of which can be operably linked to the structural coding sequence. A promoter is a DNA sequence that is capable of controlling (initiating) transcription in a cell. In some embodiments, the 5′ promoter region can include a promoter sequence that is endogenous to plants, or that is capable of naturally entering a plant cell (e.g., a sequence from a 5′ UTR that is capable of naturally entering a plant cell). For example, a promoter can be a “plant-expressible promoter” that is capable of controlling transcription in a plant cell. This includes promoters of plant origin [e.g., T-DNA gene promoters, developmental-specific promoters, tissue specific promoters (e.g., mesophyll-specific promoters), seed-specific promoters, constitutively active promoters (e.g., Ubi1, Uep1, or Act1), or organ-specific promoters (e.g., stem-, leaf-, root-, tuber-, stolon-, tricome-, ovule-, anther-, pollen-, pollen tube-, sepal-, or pistil-specific promoters)], as well as promoters of non-plant origin that are capable of directing transcription in a plant cell (e.g., promoters of viral or bacterial origin, such as the CaMV35S promoter). A promoter that is “operably linked” to a structural coding sequence can effectively control expression of the structural coding sequence. Thus, a structural coding sequence is “operably linked” and “under the control” of a promoter in a cell when RNA polymerase is able to transcribe the coding sequence into RNA.

In some embodiments, the structural coding sequence can encode a rare-cutting endonuclease, or a portion (e.g., a subunit) of a rare-cutting endonuclease. The term “rare-cutting endonuclease” refers to a natural or engineered protein that has endonuclease activity directed to nucleic acid sequences containing a recognition sequence (target sequence) that typically is about 12-40 bp in length (e.g., 14-40 bp in length; see, e.g., Baker, Nature Methods 9:23-26, 2012). Rare-cutting endonucleases generally cause cleavage inside their recognition site, leaving 2 to 4 nt staggered cut with 3′ OH or 5′ OH overhangs. Further, active rare-cutting endonucleases can be multimeric or associated with accessory molecules. Thus, rare-cutting endonucleases can be made up of subunits of monomers, accessory molecules, or combinations thereof that are required for conferring endonuclease activity at a target nucleic acid sequence.

Rare-cutting endonucleases include, for example, meganucleases, such as wild type or variant homing endonucleases [e.g., those belonging to the dodecapeptide family (LAGLIDADG (SEQ ID NO:10); see, WO 2004/067736]. Rare-cutting endonucleases also include fusion proteins that contain a DNA binding domain and a catalytic domain with cleavage activity. For example, transcription activator-like effector (TALE) endonucleases and zinc-finger-nucleases (ZFN) are fusions of DNA binding domains with the catalytic domain of the endonuclease FokI. Customized TAL effector endonucleases are commercially available under the trade name TALEN™ (Cellectis, Paris, France). Thus, the methods provided herein can include the use of TAL effector endonucleases, ZFNs, and meganucleases.

Methods for selecting endogenous target sequences and generating rare-cutting endonucleases (e.g., TALE endonucleases) targeted to such sequences can be performed as described elsewhere. See, for example, PCT Publication No. WO 2011/072246 (which is incorporated herein by reference in its entirety). TAL effectors are found in plant pathogenic bacteria in the genus Xanthomonas. These proteins play important roles in disease, or trigger defense, by binding host DNA and activating effector-specific host genes (see, e.g., Gu et al., Nature 435:1122-1125, 2005; Yang et al., Proc Natl Acad Sci USA 103:10503-10508, 2006; Kay et al. Science 318:648-651, 2007; Sugio et al., Proc Natl Acad Sci USA 104:10720-10725, 2007; and Römer et al. Science 318:645-648, 2007). Specificity depends on an effector-variable number of imperfect, typically 34 amino acid repeats (Schornack et al., J Plant Physiol 163:256-272, 2006; and WO 2011/072246). Polymorphisms are present primarily at repeat positions 12 and 13, which are referred to herein as the repeat variable-diresidue (RVD). The RVDs of TAL effectors correspond to the nucleotides in their target sites in a direct, linear fashion, one RVD to one nucleotide, with some degeneracy and no apparent context dependence. This mechanism for protein-DNA recognition enables target site prediction for new target specific TAL effectors, as well as target site selection and engineering of new TAL effectors with binding specificity for the selected sites.

TAL effector DNA binding domains can be fused to endonuclease sequences, resulting in chimeric endonucleases targeted to specific, selected DNA sequences, and leading to subsequent cutting of the DNA at or near the targeted sequences. The fact that some endonucleases (e.g., FokI) function as dimers can be used to enhance the target specificity of TALE endonucleases. For example, in some cases a pair of TALE endonuclease monomers targeted to different DNA sequences can be used. When the two TAL effector endonuclease recognition sites are in close proximity, the inactive monomers can come together to create a functional enzyme that cleaves the DNA. By requiring DNA binding to activate the nuclease, a highly site-specific restriction enzyme can be created.

In some embodiments, the methods provided herein can include the transient expression of programmable RNA-guided endonucleases, or portions (e.g., subunits) thereof. RNA-guided endonucleases are a new genome engineering tool that has been developed based on the RNA-guided CRISPR (Clustered Regularly Interspaced Short Palindromic Repeats)-associated nuclease (Cas9) from the type II prokaryotic CRISPR adaptive immune system (see, e.g., Belahj et al., Plant Methods 9:39, 2013). This system can cleave DNA sequences that are flanked by a short sequence motif known as a proto-spacer adjacent motif (PAM). Cleavage is achieved by engineering a specific CRISPR RNA (crRNA) that is complementary to the target sequence that associates with the Cas9 endonuclease. In this complex, the trans-activating crRNA (tracrRNA):crRNA complex acts as a guide RNA that directs the Cas9 endonuclease to the cognate target sequence. A synthetic single guide RNA (sgRNA) also has been developed that, on its own, is capable of targeting the Cas9 endonuclease. This tool can be expressed from a Ti, Ri, or T-DNA plasmid, as described herein, to genetically engineer plant cells. Thus, in some embodiments, the coding sequence of the Cas9 endonuclease and sgRNA or tracrRNA:crRNA can be transiently expressed from a Ti, Ri, or T-DNA plasmid as provided herein. In some embodiments, a Cas9 endonuclease coding sequence and sgRNA sequence or tracrRNA and crRNA sequence can be cloned into an iiT-DNA plasmid following the RB sequence. In some embodiments, the Cas9 endonuclease sequence and sgRNA sequence or tracrRNA and crRNA sequences can be cloned into a RRB-iiT-DNA plasmid, following the RB sequence and rare-cutting endonuclease target sequence. That is, since the RB sequence is in the 5′→3′ direction, the coding sequences can be positioned upstream of the RB: 5′-coding sequences—RB-3′ or 5′-coding sequences-rare cutting endonuclease target-RB-3′. As used herein, a “rare-cutting endonuclease target sequence” is a nucleotide sequence that is specifically recognized and cleaved by a rare-cutting endonuclease.

The expression of Cas9 can be controlled by an RNA polymerase II promoter, including but not limited to, a constitutive promoter (e.g., a Cauliflower mosaic virus (CaMV) 35S promoter, a nopaline synthase promoter, or an octopine synthase promoter), or a tissue specific or inducible promoter (e.g., a napin promoter, a phaseolin promoter, a PTA29 promoter, a PTA26 promoter, a PTA13 promoter, an XVE estradiol-inducible promoter, or an ethanol-inducible promoter). The expression of sgRNA or tracrRNA and crRNA sequence can be controlled by, for example, RNA polymerase III promoters, including, but not limited to, U6, U3 and 7SL.

In some embodiments, an iiT-DNA or RRBiiT-DNA sequence can be transferred to a plant, plant part, or plant cell. The plant can be (or the plant part or plant cell can be from), without limitation, rye, sorghum, wheat, canola, cotton, Indian mustard, sunflower, alfalfa, clover, pea, peanut, pigeonpea, red clover, soybean, tepary bean, taro, cucumber, eggplant, lettuce, tomato, carrot, cassava, potato, sweet potato, yam, Bermudagrass, perennial ryegrass, switchgrass, tall fescue, turf grasses, American elm, cork oak, eucalyptus tree, pine, poplar, rubber tree, banana, citrus, coffee, papaya, pineapple, chickpea, sugarcane, American chestnut, cabbage, apple, blueberry, grapevine, strawberry, walnut, carnation, chrysanthemum, orchids, petunia, rose, ginseng, hemp, opium poppy, Arabidopsis, oat, tobacco, and barley.

Suitable methods for transferring iiT-DNA or RRBiiT-DNA sequences to plants, plant parts, or plant cells include, for example, Agrobacterium-mediated transformation methods, including (without limitation) floral dip transformation and methods of transforming leaf explants, cotyledon explants, scutella, embryos, callus, and root explants.

In some embodiments, cells that have been contacted with Agrobacterium can be regenerated into whole plants. The whole plants then can be screened for mutations at the target sequence for the rare-cutting endonuclease. Regeneration can be achieved using established methods described elsewhere (see, for example, Shrawat et al., Plant Biotech J 4:575-603, 2006; Somers et al., Plant Physiol 131(3):892-899, 2003; Hiei et al., Plant Mol Biol 35:205-218, 1997; Vasil et al., Methods Molec Biol 111:349-358, 1999; and Jones et al., Plant Methods 1:5, 2005).

It is to be noted, however, that the structural coding sequences in the modified Ti, Ri, and T-DNA plasmids provided herein are not limited to nuclease coding sequences. In fact, any transgene sought to be transiently expressed in a susceptible plant cell (a plant cell receptive to a modified Ti, Ri, or T-DNA plasmid, as described herein) can be used.

In some embodiments, the methods provided herein can include introducing into a plant cell a modified Ti, Ri, or T-DNA plasmid having a T-DNA region that contains a T-DNA border sequence, a first sequence encoding a first polypeptide of interest, and a second sequence encoding a second polypeptide of interest. The first and second polypeptide-encoding sequences each can include a structural coding sequence that encodes a polypeptide of interest, as well as a 5′ promoter region and a 3′ non-translated region encoding a polyadenylation signal. The T-DNA border sequence can be positioned 5′ or 3′ of the polypeptide encoding sequences. The promoters in the first and second polypeptide-encoding sequence can be the same or can differ from one another. Similarly, the 3′ non-translated regions in the first and second polypeptide-encoding sequences can be the same or can differ from one another. The promoter region and the 3′ non-translated region in the first polypeptide-encoding sequence can be operably linked to the structural coding sequence encoding the first polypeptide of interest, and the promoter region and the 3′ non-translated region in the second polypeptide-encoding sequence can be operably linked to the structural coding sequence encoding the second polypeptide of interest.

In some embodiments, when the T-DNA region in the modified Ti, Ri, or T-DNA plasmid contains first and second polypeptide-encoding sequences, each polypeptide-encoding sequence can encode a rare-cutting endonuclease or a portion (e.g., a subunit) of a rare-cutting endonuclease. For example, the first and second polypeptide-encoding sequences each can contain a structural coding sequence that encodes a TAL effector endonuclease, a zinc-finger nuclease, a meganuclease, or a programmable RNA-guided endonuclease, or a portion thereof. In some cases, the rare-cutting endonucleases (or portions thereof) encoded by the first and second polypeptide-encoding sequences can be different from each other, and, upon expression in a plant cell, can work together to cleave the endogenous plant DNA at a target sequence.

In some embodiments, the methods provided herein can include introducing to a susceptible plant cell an organism that is capable of horizontal gene transfer, and that contains a modified Ti, Ri, or T-DNA plasmid with a T-DNA region as described herein. A plant cell is considered to be susceptible if it can be transformed by a T-DNA sequence as provided herein. It is noted that some plant cells may not be successfully transformed due to factors such as pattern triggered immunity, effector triggered immunity, or non-host resistances. The organism can be, for example, a bacterium (e.g., an Agrobacterium, an Ensifer, or a Rhizobium).

As described herein, infiltration of plant tissue with Agrobacterium harboring an integration-inhibited Ti, Ri, or T-DNA plasmid encoding a nuclease of interest can be used to introduce transcriptionally competent T-DNA that can be transcribed and translated, allowing the nuclease to target the site of interest. To be considered a successful event, the site of interest must be modified through non-homologous end-joining (NHEJ) or homologous recombination (HR), without T-DNA integration. Genomic DNA from regenerated tissue can be sequenced to verify site-directed mutation and lack of T-DNA integration. The lack of T-DNA integration also can be assessed using techniques such as Southern blotting, with the plasmid backbone as a probe.

In some embodiments, a modified Ti, Ri, or T-DNA plasmid can include reagents for gene targeting. As used herein, the term “gene targeting” refers to the modification of genomic DNA (e.g., eukaryotic genomic DNA) using homologous recombination. The modified Ti, Ri, or T-DNA plasmid can include a donor molecule sequence, or a donor molecule sequence and a sequence encoding a rare-cutting endonuclease that is targeted to a chromosomal sequence. The donor molecule can contain sequence that is at least about 90% homologous (e.g., about 90 to 95%, about 95 to 99%, or 100% homologous) to a sequence at or near the rare-cutting endonuclease target site in the chromosomal DNA. The donor can also include a sequence that is not homologous to the chromosomal DNA but is flanked by sequences that are at least about 90% homologous a sequence at or near the rare-cutting endonuclease target site in the chromosomal DNA. After successful gene targeting, the non-homologous sequence can be incorporated into the host genome.

In another embodiment, a genetic modification introduced by a rare-cutting endonuclease, or a rare-cutting endonuclease and donor molecule, can confer a selectable or screenable phenotype to a plant, plant part, or plant cell. The selectable phenotype can be, without limitation, herbicide tolerance or antibiotic resistance. The screenable phenotype can be, for example, expression of a fluorescent protein, expression of beta-glucuronidase, or a particular genetic modification. In some embodiments, the selectable phenotype can assist with regeneration of modified cells into whole plants.

In some embodiments, a modified Ti, Ri, or T-DNA plasmid can include a reporter sequence that can be transiently expressed with the structural coding sequence, thus facilitating determination of whether transformation was successful, and providing a screening tool for confirming that the T-DNA sequence has not integrated into the genomic DNA. Useful reporters include, without limitation, visual reporters [e.g., YFP and green fluorescent protein (GFP)], and antibiotic resistance genes (e.g., bar, pmi, nptII, als, epsps, and hph).

In some embodiments, a modified Ti, Ri, or T-DNA plasmid can include a duplicated and inverted sequence adjacent to or at the T-DNA border sequence. The duplicated and inverted target sequence can promote the formation of a stem-loop structure in single-stranded and double-stranded DNA. For example, after release from the T-DNA plasmid, the duplicated and inverted sequence can facilitate the formation of a stem-loop. This stem-loop can be unfavorable for T-DNA integration due to steric hindrance of the free DNA end. Once the single-stranded DNA is converted into a double-stranded T-DNA molecule by host polymerases, the duplicated and inverted sequence can facilitate the formation of a double-stranded stem-loop. Similar to the stem-loop in the single-stranded DNA, the double-stranded stem-loop can reduce DNA integration through steric hindrance of the free DNA ends, thereby making the T-DNA ends unfavorable for integration.

In some embodiments, a modified Ti, Ri, or T-DNA plasmid can include a rare-cutting endonuclease target site downstream of the T-DNA border sequence. This target site can allow the T-strand border sequence to be nicked by the VirD1/VirD2 complex, followed by covalent attachment of VirD2 to the border sequence, which directs the nascent T-strand to the plant cell's nucleus. Once the T-strand has entered the nucleus, the plant machinery can make the T-strand double-stranded so that it is capable of being transcribed. Transient expression of the encoded rare-cutting endonucleases can allow for site-directed mutagenesis of the plant's genomic DNA, as well as creating a double-stranded break at the rare-cutting endonuclease target site downstream of the T-DNA border sequence. Such cleavage can cause the border sequence and the covalently attached VirD2 to dissociate from the T-strand, further reducing the likelihood of integration (Mysore et al., Mol Plant-Microbe Interactions, 11(7):668-683, 1998).

Thus, this document also provides methods for transiently expressing a polypeptide in a plant cell by introducing the plant cell to an organism that is capable of horizontal gene transfer, and that contains a modified Ti, Ri, or T-DNA plasmid having a T-DNA region that includes, a T-DNA border sequence, a target site for a rare-cutting endonuclease, and a polypeptide-encoding sequence, where the rare-cutting endonuclease target site is downstream of the T-DNA border. As described herein, the polypeptide-encoding sequence can include a 5′ promoter region, a structural coding sequence encoding a polypeptide, and a 3′ non-translated region encoding a polyadenylation signal, where the 5′ promoter region and the 3′ non-translated region are operably linked to the structural coding sequence.

In some embodiments, the methods provided herein can include using a modified Ti, Ri, or T-DNA plasmid to generate genetically modified plant cells. Such methods can include introducing into a susceptible plant cell a modified Ti, Ri, or T-DNA plasmid having a T-DNA region that includes (i) a T-DNA border sequence that has been mutated (e.g., by mutation or deletion), such that the T-DNA region does not integrate into the plant cell genome, and (ii) a polynucleotide sequence encoding a rare-cutting endonuclease or rare-cutting endonuclease subunit, where the polynucleotide sequence is operably linked to a promoter that is induced in the plant cells such that the rare-cutting endonuclease or rare-cutting endonuclease subunit is transiently expressed in the plant cells. The methods also can include selecting a plant cell in which transient expression of the rare-cutting endonuclease or rare-cutting endonuclease subunit has resulted into a genome modification by specific cleavage activity. In some embodiments, the methods further can include regenerating a whole plant from a plant cell identified as having the genome modification.

Thus, the present disclosure provides general methods of gene editing, wherein a plant cell genome can be modified using T-DNA but without integration of the T-DNA into the plant cell genome. The methods generally include the steps of (a) introducing into plant cells a T-DNA that encodes a rare cutting endonuclease or endonuclease subunit and that has only one or no border functional sequences, (b) transiently expressing the rare-cutting endonuclease or endonuclease subunit in the plant cell, (c) selecting a plant cell in which a genetic modification is observed at the locus targeted by the rare-cutting endonuclease, and optionally, (d) regenerating a whole plant from the selected plant cell.

As set forth herein, new plant traits can be generated using organisms that are capable of horizontal gene transfer, such as Agrobacterium, without insertion of a transgene, especially a T-DNA transgene. Plants regenerated using the methods described herein can have rare-cutting endonuclease-induced mutations that are stably inherited, and may be cross bred with other germplasm to obtain adapted valuable new crop varieties. When a gene edition does not integrate exogenous DNA sequences (e.g., when the targeted locus is merely mutated or repaired), the resulting plants may be considered as non-GMO since they do not include foreign DNA in their genomes.

In some embodiments, the methods provided herein can further include introducing the plant cell to a second organism that is capable of horizontal gene transfer, and that contains a modified Ti, Ri, or T-DNA plasmid having a T-DNA region that includes a second T-DNA border sequence that can be identical to or differ from the first T-DNA border sequence, and a second polypeptide-encoding sequence, or a second T-DNA border sequence, a second polypeptide-encoding sequence, and a third polypeptide-encoding sequence. In such embodiments, the second and/or third polypeptide-encoding sequence(s) can include a 5′ promoter region, a structural coding sequence encoding a polypeptide, and a 3′ non-translated region encoding a polyadenylation signal, where the 5′ promoter regions and the 3′ non-translated regions are operably linked to the structural coding sequences. The second (or second and third) polypeptide-encoding sequence can be the same as or different from the polypeptide-encoding sequence contained in the modified Ti, Ri, or T-DNA plasmid of the first organism. When such methods are used, the first and second organisms can be introduced to the plant cell simultaneously (e.g., by mixing or co-culturing the first and second organisms prior to introducing them to the cell), or sequentially. For example, the first organism can be introduced to the plant cell, followed by one to five (e.g., one, two, three, four or five) days of incubation, and then the second organism can be introduced.

In addition to the methods described herein, this document also provides the modified Ti and Ri plasmids, and T-DNA plasmids, described herein. For example, this document provides modified Ti and Ri plasmids, and T-DNA plasmids, that include a T-DNA region that contains a T-DNA border sequence and a polynucleotide sequence encoding a polypeptide of interest, wherein the polypeptide-encoding sequence is operably linked to a promoter induced in a plant cell. In some embodiments, the polypeptide of interest can be a rare-cutting endonuclease (e.g., a TAL effector endonuclease, a ZFN, a meganuclease, or a programmable RNA-guided endonuclease), or a rare-cutting endonuclease subunit. In addition, in some embodiments, the Ti and Ri plasmids, and T-DNA plasmids, provided herein can further contain a target site for the rare-cutting endonuclease. The target site can be downstream of the T-DNA border sequence, for example.

This document also provides isolated host cells transformed with a modified Ti, Ri, or T-DNA plasmid, as provided herein. The host cells can be, for example, Agrobacterium cells.

Further, this document provides compositions and articles of manufacture that include one or more Ti plasmids, Ri plasmids, and/or T-DNA plasmids, as described herein, optionally in combination with packaging material and one or more additional components (e.g., buffers or other reagents) for use in the methods described herein. In some embodiments, a composition or article of manufacture can include host cells transformed with a modified Ti, Ri, or T-DNA plasmid, as provided herein. The one or more plasmids and/or the host cells can be packaged using packaging material known in the art for compositions and articles of manufacture. Further, the compositions and articles of manufacture can have a label (e.g., a tag or label secured to the packaging material, a label printed on the packaging material, or a label inserted within the package). The label can indicate that the composition(s), plasmid(s) and/or host cells contained within the package can be used to generate genetically modified plants, plant parts, or plant cells, for example.

The invention will be further described in the following examples, which do not limit the scope of the invention described in the claims.

### EXAMPLES

### Example 1

### Engineering Sequence-Specific Nucleases to Mutagenize the ALS2 Gene

To completely inactivate or knock-out the ALS2 gene in Nicotiana benthamiana, sequence-specific nucleases were designed just downstream of the protein coding sequence using software that specifically identifies TALE nuclease recognition sites, such as TALE-NT 2.0 (Doyle et al., Nucleic Acids Res 40:W117-122, 2012). The TALE nuclease recognition sites for the ALS2 genes are listed in Table 2; this TALE nuclease is designated as ALS2_T1. TALE nucleases were obtained from Cellectis Bioresearch (Paris, France).

TABLE 2
ALS2_T1 TALE nuclease
target sequences

| Target | SEQ | Target | SEQ |
|---|---|---|---|
| Sequence Left | ID: | Sequence Right | ID: |
| TAGCTTGTTCCACATTT | 14 | ACAGTACGACCCAGTCT | 15 |

### Example 2

### ALS2-T1 TALE Nuclease Activity in Yeast

To assess the activity of the TALE nucleases targeting the ALS2 genes, activity assays were performed in yeast by methods similar to those described elsewhere (Christian et al., Genetics 186:757-761, 2010). For these assays, a target plasmid was constructed with the TALE nuclease recognition site cloned in a non-functional β-galactosidase reporter gene. The target site was flanked by a direct repeat of β-galactosidase coding sequence such that if the reporter gene was cleaved by the TALE nuclease, recombination would occur between the direct repeats and function would be restored to the β-galactosidase gene. β-galactosidase activity, therefore, served as a measure of TALE nuclease cleavage activity. In the yeast assay the ALS2_T1 TALE nuclease pair displayed cleavage activity. Cleavage activities were normalized to the benchmark nuclease, I-SceI. Results are summarized in Table 3.

TABLE 3
ALS2 TALE nuclease activity in yeast

Activity in yeast*

| Target Subunit | ALS2_T1 37° C. | ALS2_T1 30° C. |
|---|---|---|
| ALS2 T01 Left | 0.97 (0.02) | 0.86 (0.02) |
| ALS2 T01 Right | | |

*Normalized to I-SceI (max = 1.0)

### Example 3

### Construction of Integration-Inhibited T-DNA Plasmid

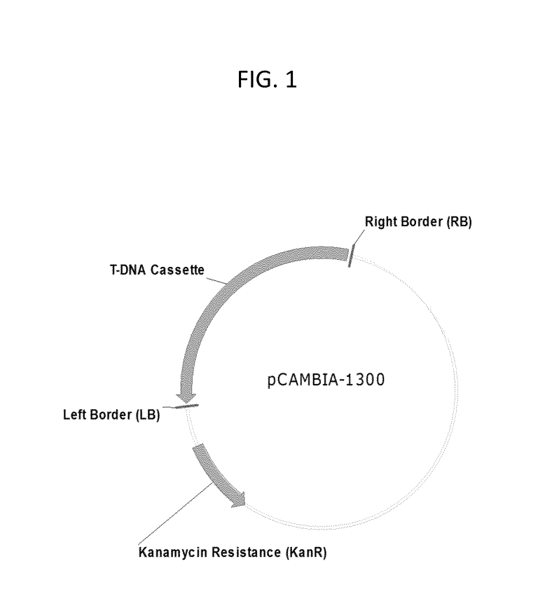

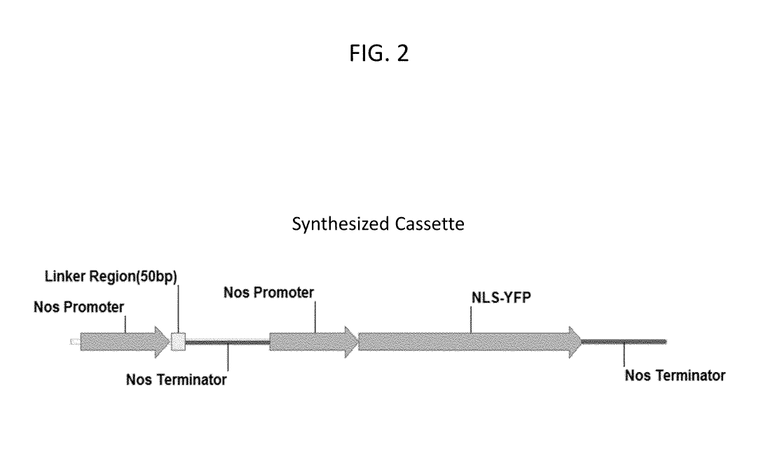

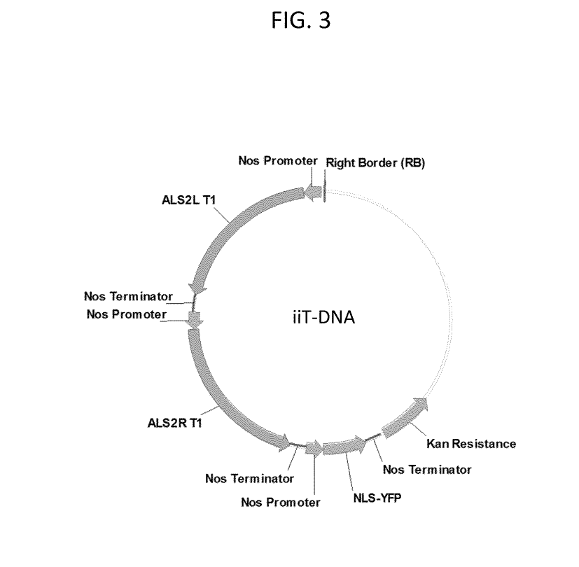

To achieve transient expression of desired nucleases sans integration of transfer DNA (T-DNA), a new vector was synthesized that lacks a LB. This modification inhibits VirD1/VirD2 border-specific endonucleases from nicking the LB, resulting in a T-DNA cassette without the proper processing required for efficient integration. To construct the integration-inhibited T-DNA (iiT-DNA) plasmid, the pCAMBIA-1300 (Cambia, Canberra, Australia) plasmid (FIG. 1) was modified using restriction enzymes to remove the left border and insert a synthesized cassette (GenScript USA Inc.) containing: (i) right border, (ii) Nos promoter, (iii) linker sequence that includes 22 restriction sites for directional TALE nuclease subunit A cloning, (iv) Nos terminator, (v) restriction site for TALE nuclease subunit B (TALE nuclease subunit B cassette contains Nos promoters and Nos terminators) cloning purposes, (vi) Nos promoter, (vii) yellow fluorescence protein with nuclear localization signal, and a (viii) Nos terminator (FIG. 2). This cassette was synthesized for ligation into the modified pCAMBIA-1300 utilizing the IN-FUSION® HD Cloning Kit (Clontech Laboratories, Inc.). After verification in E. coli, this plasmid was subjected to restriction enzyme digests followed by ligations of the TALE nuclease subunits to yield the desired product (FIG. 3), at which point it was transformed into Agrobacterium tumefaciens.

### Example 4

### Transient Expression of YFP Via iiT-DNA Plasmid

To demonstrate the ability of the iiT-DNA plasmid to transiently express a desired protein without integration, YFP is transformed into N. benthamiana and monitored over a twenty day period. An accelerated decrease of fluorescence in the iiTi treatment is indicative of transient expression. This demonstration is accomplished by needleless syringe infiltration of A. tumefaciens (containing the two aforementioned constructs) into N. benthamiana whole leaves. The fluorescence expression levels of the transformed leaves are followed over a time course of twenty days. These images are quantified using the Cell Profiler (Broad Institute) software, which allows relative fluorescence units (RFU) to be compared between the iiT-DNA and control plasmids. The reduction of integration is confirmed by a much steeper decrease in YFP fluorescence throughout the time course in the cells inoculated with iiT-DNA plasmids, as well as the lack of stable expression of YFP fluorescence at approximately 9 dpt.

### Example 5

### Transient Expression of ALS2 TALE Nuclease Via iiT-DNA Plasmid

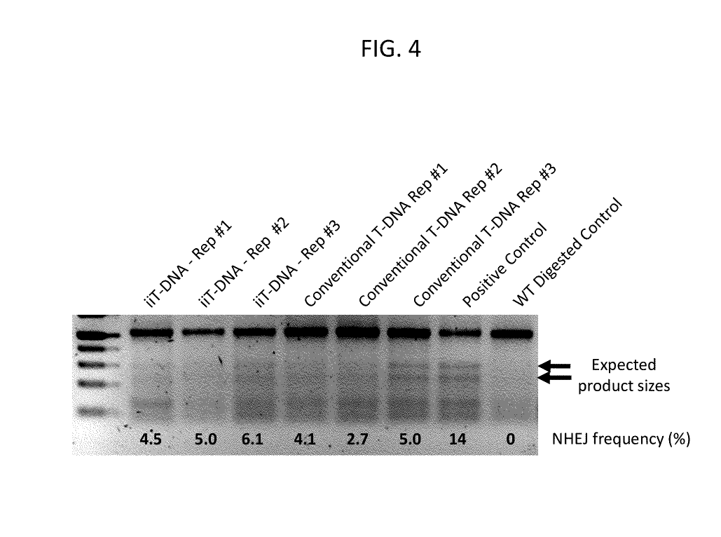

To demonstrate transient expression of a nuclease resulting in site-directed mutagenesis sans integration, N. benthamiana whole leaves were infiltrated with A. tumefaciens using a needleless syringe. Two strains of A. tumefaciens were tested: one containing an iiT-DNA plasmid encoding the ALS2 TALE nuclease, and the other containing a conventional T-DNA plasmid encoding the same TALE nuclease. By directly comparing NHEJ frequencies between the different A. tumefaciens strains, it was possible to indirectly measure the relative T-DNA transfer efficiency. Two days post infiltration of N. benthamiana leaves, genomic DNA was isolated and the ALS gene was amplified by PCR. The resulting PCR product was subjected to T7 endonuclease I digestion. NHEJ frequencies were quantified based on band intensity using the calculation NHEJ frequency=100×(1−(1−fraction cleaved)̂½). Surprisingly, similar mutation frequencies were observed for the samples containing the iiT-DNA and the samples containing conventional T-DNA (FIG. 4). These data indicated that transfer is not impaired when using iiT-DNA plasmids.

### Example 6

### Transient Expression of ALS2 TALEN Via Integration-Inhibited Ti Plasmid Utilizing a Stem-Loop Structure Near the Right Border

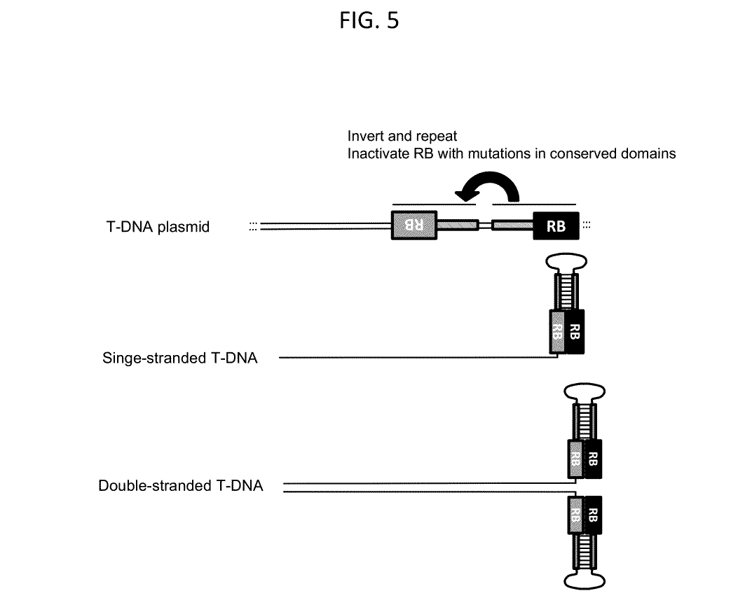

To demonstrate transient expression of a nuclease resulting in site-directed mutagenesis sans integration utilizing a stem-loop structure near the right border (e.g., within about 1000 nucleotides of the right border; FIG. 5), N. benthamiana whole leaves are infiltrated via a needleless syringe with A. tumefaciens (as described above), and NHEJ and integration frequencies are compared between the stem-loop iiT-DNA and control plasmids. These data are obtained after taking leaf discs from the infiltrated regions of the whole leaves 7 dpt to survey NHEJ frequency via 454 deep sequencing, and T-DNA integration by qRT-PCR.

### Example 7

### Validation of Reduced Integration with iiT-DNA Plasmids in Comparison with Conventional T-DNA Plasmids

To demonstrate that removal of the LB decreases the frequency of stable integration as compared to a conventional T-DNA plasmid, Nicotiana tabacum cotyledons were transformed by Agrobacterium using the floral dip method (Clough and Bent, Plant J, 16:735-743, 1998). Two strains of A. tumefaciens were tested: one containing an iiT-DNA plasmid encoding a kanamycin selectable marker, and the other containing a conventional T-DNA plasmid encoding the same kanamycin selectable marker. Unlike the iiT-DNA plasmid, however, the conventional T-DNA plasmid contained a unique KpnI restriction site downstream of the kanamycin stop codon, thereby permitting identification of conventional T-DNA sequence after integration into the plant genome. The two different Agrobacterium strains were grown to an OD₆₀₀=0.6, at which point the resuspended cultures were mixed in a 1:1 ratio. This mixture was then used to transform Nicotiana tabacum cotyledons using standard transformation protocols (Horsch et al., Science, 227:1229-1231, 1985). Transformed cotyledons were grown on selective regeneration medium for 6-8 weeks under kanamycin selection until shoots regenerated, at which point the shoot tissue was sacrificed and subjected to DNA extraction. The extracted DNA was then used in a PCR designed to amplify the NptII resistance gene. The resulting amplicons were subjected to a KpnI restriction enzyme digest, allowing for high-throughput screening of individual transformation events for determining which T-DNA was integrated into the host genome. Using this method, about 10-fold lower integration events were observed with the iiT-DNA, as compared to the conventional T-DNA, indicating that removal of the LB sequence effectively inhibited T-DNA integration. Results are summarized in Table 4.

TABLE 4
Integration frequency of the iiT-DNA vector

| T-DNA within the plant genome | Events (#) | Event (%) |
|---|---|---|
| iiT-DNA | 14 | 5.8 |
| conventional T-DNA | 165 | 67.9 |
| iiT-DNA + conventional T-DNA | 64 | 26.3 |

### Example 8

### Transient Expression of ALS2 TALEN Via a iiT-DNA Plasmid Utilizing a Removable Right Border

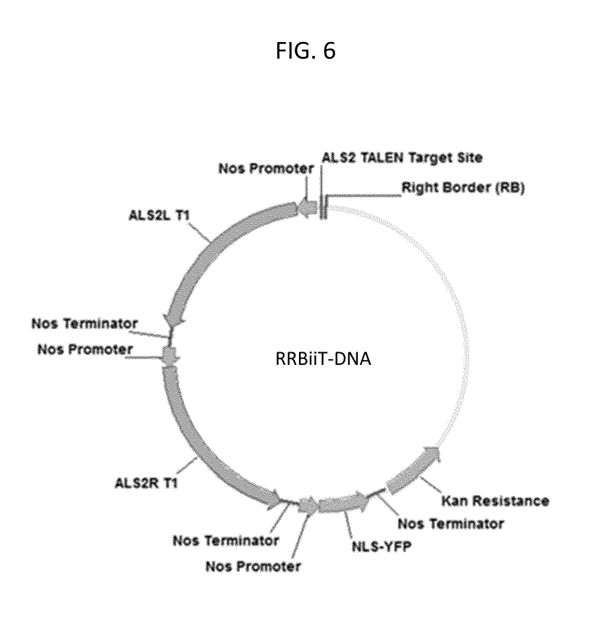

To demonstrate transient expression of a nuclease resulting in site-directed mutagenesis sans integration utilizing a removable RB (FIG. 6), N. benthamiana whole leaves are infiltrated via a needleless syringe using A. tumefaciens (as described above), and NHEJ and integration frequencies are compared between the removable right border (RRB)-iiT-DNA and control plasmids. These data are obtained after taking leaf discs from the infiltrated regions of the whole leaves 7 dpt to survey NHEJ frequency via 454 deep sequencing, and T-DNA integration by qRT-PCR.

### Example 9

### Reduced Integration of the iiT-DNA

To demonstrate that removal of the LB decreases the frequency of stable integration as compared to a conventional T-DNA plasmid, Arabidopsis is transformed using an Agrobacterium floral dip method. To determine the integration frequency, two different Agrobacterium strains are grown to an OD₆₀₀=0.6, at which point the resuspended cultures are mixed in a 1:1 ratio. This mixture is used to transform the Arabidopsis thaliana via a floral dip method. Plants grow for another 3-5 weeks until the siliques have dried, at this point the seeds are harvested and grown in agar with kanamycin to select for only seeds that have been transformed. Resistant seeds are then grown and genotyped to determine which plasmid, iiT-DNA or conventional T-DNA, is responsible for the resistance. The iiT-DNA plasmid should exhibit a lower integration frequency than the conventional -T-DNA plasmid.

### Example 10

### Reduced Integration of the Integration-Inhibited iiT-DNA Plasmid Utilizing a Stem-Loop Structure Adjacent to the Right Border

To demonstrate that a stem-loop structure near the RB sequence (e.g., within about 1000 nucleotides of the RB) decreases the frequency of stable integration as compared to a conventional T-DNA plasmid, Arabidopsis is transformed using an Agrobacterium floral dip method. To determine the integration frequency, two different Agrobacterium strains are grown to an OD₆₀₀=0.6, at which point the resuspended cultures are mixed in a 1:1 ratio. This mixture is used to transform the Arabidopsis thaliana via a floral dip method. Plants grow for another 3-5 weeks until the siliques have dried, at this point the seeds are harvested and grown in agar with kanamycin to select for only seeds that have been transformed. Resistant seeds are then grown and genotyped to determine which plasmid, stem-loop iiT-DNA or conventional T-DNA, is responsible for the resistance. The stem-loop iiT-DNA plasmid should exhibit a lower integration frequency than the conventional T-DNA plasmid.

### Example 11

### Reduced Integration of the iiT-DNA Utilizing a Removable Right Border

To demonstrate that the removal of the RB, through cleavage of the iiT-DNA by a sequence-specific nuclease, decreases the frequency of stable integration as compared to a conventional T-DNA plasmid, Arabidopsis was transformed using an Agrobacterium floral dip method. The removable RB iiT-DNA is designated as RRBiiT-DNA. To determine the integration frequency, two different Agrobacterium strains were tested: one containing an RRB iiT-DNA encoding a TALE nuclease and a kanamycin selectable marker, and the other containing a conventional T-DNA plasmid encoding the same kanamycin selectable marker, but with a unique KpnI restriction digestion sequence. The Agrobacterium strains were grown to an OD₆₀₀=0.6, at which point the resuspended cultures were mixed in a 1:1 ratio. This mixture was used to transform Arabidopsis via the floral dip method. Plants were grown for another 3-5 weeks until the siliques have dried, at which point the seeds were harvested and grown in agar with kanamycin to select for only seeds that have been transformed. Resistant seeds were then grown and genotyped to determine which 5 plasmid, RRB iiTi or conventional T-DNA plasmid, was responsible for the resistance. Of nine independent events, nine plants contained the conventional T-DNA and no plants contained the RRBiiT-DNA. Thus, the RRBiiT-DNA plasmid exhibited a lower integration frequency than the conventional T-DNA plasmid.

TABLE 5
Integration frequency of the RRBiiT-DNA

| T-DNA within the plant genome | Events (#) | Event (%) |
|---|---|---|
| RRBiiT-DNA | 0 | 0 |
| conventional T-DNA | 9 | 100 |
| RRBiiT-DNA + conventional T-DNA | 0 | 0 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for transiently expressing a polypeptide in a plant cell, the method comprising introducing into the plant cell a modified Ti, Ri, or T-DNA plasmid, wherein the modified Ti, Ri, or T-DNA plasmid comprises a T-DNA region that comprises:
(a) a T-DNA border sequence; and
(b) a polypeptide-encoding sequence comprising a 5′ promoter region, a structural coding sequence encoding the polypeptide, and a 3′ non-translated region comprising a polyadenylation signal, wherein the 5′ promoter region and the 3′ non-translated region are operably linked to the structural coding sequence,
such that the polypeptide-encoding sequence is transiently expressed in the plant cell and does not integrate into the genome of the plant cell.

2. (canceled)

3. The method of claim 1, wherein the modified Ti, Ri, or T-DNA plasmid comprises only one functional T-DNA border sequence.

4-5. (canceled)

6. The method of claim 1, wherein the introducing step comprises contacting a susceptible plant cell with an organism capable of horizontal gene transfer.

7. (canceled)

8. The method of claim 6, wherein the organism capable of horizontal gene transfer is an Agrobacterium.

9. The method of claim 1, wherein the T-DNA border sequence is from Agrobacterium.

10. The method of claim 1, wherein the T-DNA border sequence is a T-DNA right border sequence.

11-13. (canceled)

14. The method of claim 1, wherein the T-DNA border sequence is 5′ of the polypeptide-encoding sequence in the modified Ti, Ri, or T-DNA plasmid.

15-17. (canceled)

18. The method of claim 1, wherein the polypeptide-encoding sequence encodes a rare-cutting endonuclease or rare-cutting endonuclease subunit.

19. The method of claim 18, wherein the rare-cutting endonuclease is a transcription activator-like (TAL) effector endonuclease, a zinc-finger nuclease, a meganuclease, or a programmable RNA-guided endonuclease.

20. The method of claim 18, wherein transient expression of the rare-cutting endonuclease results in site-directed mutagenesis.

21. The method of claim 1, wherein the modified Ti, Ri, or T-DNA plasmid contains a reporter gene that is transiently expressed with the structural coding sequence.

22. (canceled)

23. The method of claim 1, wherein the T-DNA region further comprises a donor sequence.

24. The method of claim 23, wherein transient delivery of the donor sequence results in gene targeting.

25. The method of claim 1, wherein the T-DNA region further comprises a second polypeptide-encoding sequence comprising a 5′ promoter region, a structural coding sequence encoding a second polypeptide, and a 3′ non-translated region encoding a polyadenylation signal, wherein the 5′ promoter region and the 3′ non-translated region are operably linked to the structural coding sequence, such that the second polypeptide-encoding sequence is transiently expressed in the plant cell and does not integrate into the genome of the plant cell.

26-28. (canceled)

29. The method of claim 25, wherein the polypeptide-encoding sequence encodes a rare-cutting endonuclease or rare-cutting endonuclease subunit, and the second polypeptide-encoding sequence encodes a second rare-cutting endonuclease or rare-cutting endonuclease subunit.

30. The method of claim 29, wherein the second rare-cutting endonuclease is a TAL effector endonuclease, a zinc-finger nuclease, a meganuclease, or a programmable RNA-guided endonuclease.

31-33. (canceled)

34. A method for generating a plant, comprising providing a plant cell obtained according to the method of claim 25, wherein the polypeptide-encoding sequence encodes a rare-cutting endonuclease or rare-cutting endonuclease subunit, and the second polypeptide-encoding sequence encodes a second rare-cutting endonuclease or rare-cutting endonuclease subunit, and regenerating the plant cell into a plant.

35. The method of claim 34, wherein the regenerated plant contains one or more mutations generated by transient expression of the rare-cutting endonucleases or rare-cutting endonuclease subunits.

36. A method for transiently expressing a polypeptide in a plant cell, the method comprising introducing a plant cell to an organism capable of horizontal gene transfer, wherein the organism contains a modified Ti, Ri, or T-DNA plasmid comprising a T-DNA region that comprises:
(a) a T-DNA border sequence;
(b) a target site for a rare-cutting endonuclease; and
(c) a polypeptide-encoding sequence comprising a 5′ promoter region, a structural coding sequence encoding the polypeptide, and a 3′ non-translated region comprising a polyadenylation signal, wherein the 5′ promoter region and the 3′ non-translated region are operably linked to the structural coding sequence,
such that the polypeptide-encoding sequence is transiently expressed in the plant cell and does not integrate into the genome of the plant cell.

37. (canceled)

38. The method of claim 36, wherein the organism capable of horizontal gene transfer is an Agrobacterium.

39. (canceled)

40. The method of claim 36, wherein the T-DNA border sequence is a T-DNA right border sequence.

41-45. (canceled)

46. The method of claim 36, wherein the polypeptide-encoding sequence encodes a rare-cutting endonuclease or rare-cutting endonuclease subunit.

47. (canceled)

48. The method of claim 46, wherein transient expression of the rare-cutting endonuclease results in site-directed mutagenesis.

49. The method of claim 36, wherein the T-DNA region further comprises a donor sequence.

50. The method of claim 49, wherein transient delivery of the donor sequence results in gene targeting.

51-53. (canceled)

54. The method of claim 36, wherein the T-DNA region further comprises a second polypeptide-encoding sequence comprising a 5′ promoter region, a structural coding sequence encoding a second polypeptide, and a 3′ non-translated region encoding a polyadenylation signal, wherein the 5′ promoter region and the 3′ non-translated region are operably linked to the structural coding sequence, such that the second polypeptide-encoding sequence is transiently expressed in the plant cell and does not integrate into the genome of the plant cell.

55-57. (canceled)

58. The method of claim 54, wherein the polypeptide-encoding sequence encodes a rare-cutting endonuclease or rare-cutting endonuclease subunit, and the second polypeptide-encoding sequence encodes a second rare-cutting endonuclease or rare-cutting endonuclease subunit.

59. (canceled)

60. The method of claim 58, wherein transient expression of the rare-cutting endonucleases or rare-cutting endonuclease subunits results in site-directed mutagenesis.

61. The method of claim 36, further comprising introducing the plant cell to a second organism capable of horizontal gene transfer, wherein the second organism contains a second modified Ti, Ri, or T-DNA plasmid comprising a T-DNA region that comprises:
(a) a T-DNA border sequence;
(b) a second polypeptide-encoding sequence comprising a 5′ promoter region, a structural coding sequence encoding a polypeptide, and a 3′ non-translated region comprising a polyadenylation signal, wherein the 5′ promoter region and the 3′ non-translated region are operably linked to the structural coding sequence,
such that the second polypeptide-encoding sequence is transiently expressed in the plant cell and does not integrate into the genome of the plant cell.

62-65. (canceled)

66. The method of claim 61, wherein the polypeptide-encoding sequence encodes a rare-cutting endonuclease or rare-cutting endonuclease subunit, and the second polypeptide-encoding sequence encodes a second rare-cutting endonuclease or rare-cutting endonuclease subunit.

67-69. (canceled)

70. The method of claim 36, further comprising introducing to the plant cell a second organism capable of horizontal gene transfer, wherein the second organism contains a modified Ti, Ri, or T-DNA plasmid comprising a T-DNA region that comprises:
(a) a T-DNA border sequence;
(b) a second polypeptide-encoding sequence comprising a 5′ promoter region, a structural coding sequence encoding the second polypeptide, and a 3′ non-translated region comprising a polyadenylation signal, wherein the 5′ promoter region and the 3′ non-translated region are operably linked to the structural coding sequence; and
(c) a third polypeptide-encoding sequence comprising a 5′ promoter region, a structural coding sequence encoding the third polypeptide, and a 3′ non-translated region encoding a polyadenylation signal, wherein the 5′ promoter region and the 3′ non-translated region are operably linked to the structural coding sequence,
such that the second and third polypeptide-encoding sequences are transiently expressed in the plant cell and are not integrated into the genome of the plant cell.

71. (canceled)

72. The method of claim 70, wherein the second polypeptide-encoding sequence encodes a second rare-cutting endonuclease or rare-cutting endonuclease subunit, and the third polypeptide-encoding sequence encodes a third rare-cutting endonuclease or rare-cutting endonuclease subunit.

73-77. (canceled)

78. A method for generating a plant, comprising providing a plant cell obtained according to the method of claim 36, wherein the polypeptide-encoding sequence encodes a rare-cutting endonuclease or a rare-cutting endonuclease subunit, and regenerating the plant cell into a plant.

79. The method of claim 78, wherein the regenerated plant contains one or more mutations generated by transient expression of the rare-cutting endonuclease.

80. A method for generating a plant, comprising providing a plant cell obtained according to the method of claim 54, wherein the polypeptide-encoding sequence encodes a rare-cutting endonuclease or rare-cutting endonuclease subunit, and the second polypeptide-encoding sequence encodes a second rare-cutting endonuclease or rare-cutting endonuclease subunit, and regenerating the plant cell into a plant.

81. The method of claim 80, wherein the regenerated plant contains one or more mutations generated by transient expression of the rare-cutting endonucleases or rare-cutting endonuclease subunits.

82. A modified Ti, Ri, or T-DNA plasmid comprising a T-DNA region that comprises:
i) only one T-DNA border sequence; and
ii) a polynucleotide sequence encoding a rare-cutting endonuclease or one or more rare-cutting endonuclease subunits, operably linked to a promoter capable of being induced in a plant cell.

83. (canceled)

84. The modified Ti, Ri, or T-DNA plasmid of claim 82, wherein the T-DNA contains a duplicated and inverted sequence adjacent to the border sequence.

85. The modified Ti, Ri, or T-DNA plasmid of claim 82, wherein the rare-cutting endonuclease or rare-cutting endonuclease subunits are from a TAL effector endonuclease, a zinc-finger nuclease, a meganuclease, or a programmable RNA-guided endonuclease.

86. The modified Ti, Ri, or T-DNA plasmid of claim 82, wherein the plasmid further comprises a target site for the rare-cutting endonuclease, and wherein the target site is downstream of the T-DNA border sequence.

87. An article of manufacture comprising the modified Ti, Ri, or T-DNA plasmid of claim 82.

88. A composition comprising the modified Ti, Ri, or T-DNA plasmid of claim 82.

89. An isolated host cell comprising the modified Ti, Ri, or T-DNA plasmid of claim 82.
